(19) United States
(12) Patent Application Publication
(10) Pub. No.: US 2021/0308040 A1
(43) Pub. Date: Oct. 7, 2021

(54) KETAMINE ORAL TRANSMUCOSAL DELIVERY SYSTEM

(71) Applicant: GUANGZHOU DAZHOU BIOMEDICINE LTD., Guangzhou (CN)

(72) Inventors: Hock S. TAN, East Brunswick, NJ (US); Huadong TANG, McLean, VA (US); Michael MAYERSOHN, Marana, AZ (US); Wenfei LIANG, Guangzhou (CN)

(21) Appl. No.: 17/288,156

(22) PCT Filed: Oct. 23, 2019
(86) PCT No.: PCT/US2019/057589

§ 371 Date: Apr. 23, 2021

Related U.S. Application Data
(60) Provisional application No. 62/751,101, filed on Oct. 26, 2018.

(51) Int. Cl. A61K 9/00; A61K 9/70; A61K 31/135; A61K 47/22; A61K 47/32; A61K 47/10; A61K 47/12; A61K 47/38

(52) U.S. Cl. CPC A61K 9/006; A61K 9/7007; A61K 31/135; A61K 47/38; A61K 47/32; A61K 47/10; A61K 47/12; A61K 47/22

(57) ABSTRACT

Provided herein are film compositions comprising ketamine suitable for delivering ketamine across oral mucosal membranes. The film compositions can have various designs such as quick-release film, slow-release film, bilayer film that combines both a quick-release and slow-release film, or one-directional release film. Further are included methods of using the film compositions herein for the treatment of various diseases or disorders, such as pain and/or depression.

### CROSS REFERENCE TO RELATED APPLICATIONS

This application claims benefit to U.S. Provisional Application No. 62/751,101, filed Oct. 26, 2018, the content of which is herein incorporated by reference in its entirety.

### BACKGROUND OF THE INVENTION

### Field of the Invention

In various embodiments, the present disclosure is generally related to ketamine compositions for delivery through oral mucosal tissues, methods of preparing and methods of using ketamine compositions, for example, in treating pain and/or depression.

### Background Art

Major depressive disorder (MDD) is a disabling psychiatric illness. Lifetime prevalence of MDD is approximately 16%. Kessler et al., JAMA, 289(23):3095-105 (2003). There are three primary classes of antidepressants that are commonly prescribed for MDD: (1) monoamine oxidase inhibitors (MAOIs); (2) tricyclics; and (3) serotonin-norepinephrine reuptake inhibitors (SNRIs) and selective serotonin reuptake inhibitors (SSRIs). There are significant limitations with the use of current antidepressants, including limited efficacy, delayed onset of action, and adverse side effects. Antidepressants have been found to be only about 20-30% more effective than placebo. The delay of onset varies from weeks to months, which may result in adverse events, including but not limited to increased vulnerability for suicide, decrease in compliance, and increase in social and economic burden. Common side effects of these antidepressants include nausea, insomnia, anxiety, weight-loss/gain, drowsiness, headache, loss of sex drive, and/or blurred vision. Penn and Tracey, Ther. Adv. Psychopharmacol., 2(5): 179-188 (2012).

Pain can present as a disabling physical illness. One type of pain, neuropathic pain, is a complex chronic pain state often accompanied by tissue injury. The occurrence of pain with neuropathic characteristics is about 6.9-10% of the general population. Hecke et al., Pain, 155(4):654-62 (2014). Symptoms of neuropathic pain include spontaneous burning, shooting pain, hyperalgesia, and allodynia. Patients with neuropathic pain often have conditions that are associated with other significant health issues, including depression, sleep problems, and loss of independence. Bouhassira et al., Pain, 136(3):380-7 (2008). Neuropathic pain can be caused by a variety of mechanisms, including infection, central or peripheral nerve injury, stroke, multiple sclerosis, diabetes mellitus, sarcoidosis, toxic agents (e.g., alcohol, chemotherapy), inherited or genetic neuropathy, and Complex Regional Pain Syndrome (CRPS). CRPS is an intractable form of pain, often resistant to a variety of conventional therapies. Correll et al., Pain Med., 5(3):263-75 (2004). Neuropathic pain is difficult to treat, with only about 40-60% of patients achieving partial relief. Treatment for neuropathic pain includes antidepressants, anticonvulsants, and/or topical pain management medications. Niesters et al., Expert Opin. Drug Metab. Toxicol., 8(11):1409-17 (2012); Dworkin et al., Pain, 132(3):237-51 (2007).

Ketamine can act as a non-competitive, N-methyl-D-aspartate (NMDA) receptor antagonist, and has been indicated, for example, for treatment as an anesthetic, sedative, and analgesic. For example, ketamine has been demonstrated to be an effective antidepressant, with rapid onset (within about 2 hours of administration) and sustained antidepressant effect (from days to, in some cases, a week or two after administration). Berman et al., Biol. Psychiatry, 47(4):351-54 (2000). The NMDA receptor pathway also plays an important role in pain, including for example neuropathic pain. Animal studies and human clinical studies have shown the efficacy of ketamine in the treatment of chronic neuropathic pain. Correll et al., Pain Med. 5(3):263-75 (2004); Sigtermans et al., Pain, 145(3):304-11 (2009).

Despite the wide range of possible indications, existing ketamine formulations and/or methods of treatments have various drawbacks. Oral tablet or capsule is typically a convenient route for the patient, however, the metabolic and pharmacokinetic properties of ketamine make such conventional oral administration less suitable. Ketamine has a high systemic (primarily hepatic) clearance of about 19 ml/min·kg, a rate, which approaches liver plasma flow. Thus, ketamine is subject to substantial pre-systemic metabolism, or first-pass effect, in the liver and gut wall by metabolic enzymes, such as cytochrome P450 enzymes (CYP450). Consequently, the absolute oral bioavailability of ketamine in humans is only about 10-20%. Due to this first-pass effect, there is an increased risk for drug-drug interactions (DDI) with drugs that can inhibit or induce CYP450s. Clements et al., J Pharm Sci, 71(5):539-42 (1981); Fanta et al., Eur. J. Clin. Pharmacol., 71:441-47 (2015); Peltoniemi et al., Basic & Clinical Pharmacology & Toxicology, 111:325-332 (2012).

### BRIEF SUMMARY OF THE INVENTION

In various embodiments, the present invention provides a drug delivery system that delivers ketamine through oral mucosal tissues, minimizing pre-systemic metabolism. In some embodiments, the present invention provides oral films, which can adhere to oral mucosal tissues within the oral cavity (e.g., sublingual mucosa or buccal mucosa). The films can be formulated in various forms, such as a quick-release (QR) film, slow-release (SR) film, a bilayer film comprising both a QR and SR film, or a one-directional release film. In some embodiments, the oral films can be a bilayer film, which can provide a biphasic pharmacokinetic (PK) profile. For example, in some embodiments, the bilayer film comprises an outer layer that is fast-dissolving, and an inner layer that is slow-dissolving. Without wishing to be bound by theories, it is believed that the outer layer, upon contacting the oral mucosal tissues in the presence of saliva, can rapidly release ketamine for absorption through the oral mucosal tissues. Thus, such a bilayer film can provide fast ketamine exposure and a fast on-set of effects, e.g., analgesia and/or antidepressant effects. The inner layer, being a slow-dissolving and sustained formulation, can then provide a sustained release of ketamine, for example, to maintain the analgesic or antidepressant effect for a prolonged time. In some embodiments, a fast-dissolving film and a slow-dissolving film can be co-packaged in a kit. In some embodiments, the fast-dissolving film, when applied, e.g., sublingually, can achieve a fast absorption and rapid on-set of intended pharmacologic action, and the slow-dissolving film, when applied, e.g., via the buccal site, can achieve a relatively long and sustained release of ketamine to maintain a longer therapeutic effect. In some embodiments, the kit can comprise various fast-dissolving and slow-dissolving films in various combinations, which can be applied, for example, to the buccal and/or sublingual mucosa to achieve a desired therapeutic effect, e.g., for the treatment of acute and chronic pain, and MDD.

In some embodiments, the present disclosure provides a slow-release film composition (or simply SR film) comprising ketamine in a mucoadhesive polymer. The SR film typically is suitable for use to deliver ketamine through an oral mucosal membrane, e.g., sublingual mucosal or buccal mucosal membrane. In some embodiments, the SR film, when tested in vitro using cultured human buccal tissue in a Franz model, delivers ketamine through the human buccal tissue at a rate of about at least 0.1 mg per cm² per hour, for example, from about 0.1 mg/cm²*h to about 5 mg/cm²*h or about 0.1 mg/cm²*h to about 2 mg/cm²*h (e.g., about 0.1 mg/cm²*h to about 1 mg/cm²*h, or about 0.2 mg/cm²*h to about 0.5 mg/cm²*h) for the first hour, first two hours, or first four hours. In some embodiments, the ketamine is present in an amount of about 5% to about 30% by weight of the SR film. In some embodiments, the ketamine is substantially in its free base form. For example, in some embodiments, the ketamine used for preparing the SR film can be ketamine base.

Suitable mucoadhesive polymers are exemplified herein. In some embodiments, the mucoadhesive polymer comprises one or more polymers selected from cellulose derivatives, polyacrylic acids, polyacrylates, polyethylene oxides, polyvinyl pyrrolidones, polyvinyl alcohols, tragacanth, alginates, gum (including karaya gum, guar gum, xanthan gum), soluble starch, gelatin, lectin, pectin, and chitosan. In some embodiments, the mucoadhesive polymer comprises one or more polymers selected from a hydrophilic polymer, a polysaccharide and its derivatives, and a hydrogel. In some embodiments, the mucoadhesive polymer comprises one or more polymers selected from polyacrylic acids, polyacrylates, celluloses, e.g., carboxy celluloses (e.g., sodium carboxymethyl cellulose), hydroxyalkyl cellulose (e.g., hydroxypropylcellulose, hydroxyethylcellulose and hydroxyethyl ethyl cellulose)), polyvinylpyrrolidone, and polyvinyl alcohol. In some embodiments, the mucoadhesive polymer comprises one or more polymers selected from Carbopol (polyacrylic acid), carboxymethyl cellulose, carboxyethyl cellulose, hydroxypropyl cellulose, hydroxypropylmethyl cellulose, and gum. In some embodiments, the mucoadhesive polymer is water-swellable. Typically, the mucoadhesive polymer is present in an amount of about 15% to about 60% by weight of the film composition.

The SR film compositions can further include a permeation enhancer and/or an antioxidant. For example, in some embodiments, the SR film composition comprises a permeation enhancer, e.g., comprising one or more permeation enhancers selected from dimethyl sulfoxide (DMSO), oleic alcohol, oleic acid, oleyl oleate, levulinic acid, propylene glycol, dipropylene glycol, ethanol, and surfactants. In some embodiments, the permeation enhancer is present in an amount of about 5% to about 30% by weight of the film composition. In some embodiments, the film composition comprises an antioxidant, e.g., tocopherol acetate. Other suitable ingredients that can be included in the SR film compositions are exemplified herein.

In some embodiments, the SR film compositions can be combined, e.g., laminated, with a quick-release (QR) film to form a bilayer or multilayer film composition. Typically, such a bilayer or multilayer film can provide a bi-phasic release profile, which can be advantageous in certain situations. In some embodiments, the quick-release film comprises ketamine and a water-soluble polymer. In some embodiments, the quick-release film is characterized in that it releases substantially all of the ketamine (e.g., at least 85%, at least 90%, or at least 95%) therein in less than 30 minutes, when tested in vitro in a dissolution test using USP Apparatus 2. In some embodiments, the water soluble polymer in the quick-release film comprises one or more polymers selected from hydroxyl propyl methyl cellulose (HPMC), hydroxylpropyl cellulose (HPC), Povidone, polyvinyl alcohols (PVA), low molecular weight polyethylene oxide (PEO such as PolyOx N10 supplied by Dow Chemical), and starch-based polymers (Lycoat, manufactured by Roquette). In some embodiments, the QR film can also optionally include a permeation enhancer, e.g., one or more permeation enhancers selected from dimethyl sulfoxide (DMSO), oleic alcohol, oleic acid, oleyl oleate, levulinic acid, propylene glycol, dipropylene glycol, ethanol, and surfactants. In some embodiments, the QR film can also optionally include an antioxidant, such as tocopherol acetate. In some embodiments, the ketamine in the QR film is substantially in its free base form.

In some embodiments, the bilayer or multilayer film can be characterized in that, when tested in an in vitro release test, it releases ketamine at a rate of at least 0.5 mg/hour/cm² (e.g., about 1 mg/hour/cm² to about 20 mg/hour/cm²) for the first hour or two hours, and releases ketamine at a rate of about 0.5 mg/hour/cm² to about 5 mg/hour/cm² thereafter for an extended period of about 2 hours, 4 hours, 6 hours, or 8 hours, or longer.

The film compositions herein (SR, QR or bilayer films) can also be optionally combined with a water-insoluble but ingestible layer to form a one-directional release film. In some embodiments, the water-insoluble but ingestible layer comprises a cellulose (e.g., ethyl cellulose), a polyacrylate (e.g., polymethylacrylate), or a combination thereof.

In some embodiments, a packaging system is provided, which can include one or more film compositions herein, which can be the same or different. For example, in some embodiments, the package can include two or more QR films herein, which can be the same or different. In some embodiments, the package can include one or more QR films and one or more SR films, which can include the same or different amount of ketamine.

In some embodiments, the present disclosure also provides a method of administering ketamine to a subject in need thereof. In some embodiments, the present disclosure provides a method of treating pain and/or depression in a subject in need thereof. In some embodiments, the method comprises administering to the subject one or more films herein.

### BRIEF DESCRIPTION OF THE DRAWINGS/FIGURES

FIG. 1 shows four exemplary oral film designs.

FIG. 2 shows an example of drug release profile of an exemplary bilayer ketamine film.

FIG. 3 shows drug permeability studies of films of Example 2 through cultured human buccal tissues in a Franz model.

FIG. 4A and FIG. 4B show examples of packaging for multiple films.

### DETAILED DESCRIPTION OF THE INVENTION

Oral mucoadhesive films, also referred to as orally dissolving or eroding thin films or oral bioadhesive films, have emerged as an advanced alternative dosage form to the more traditional oral ingested forms including tablets, capsules, soft gels and liquids often associated with prescription and OTC (over-the-counter) medications. The thin film strips, squares or discs, containing active pharmaceutical ingredient (API), are typically designed for intra-oral administration, with the patient placing the strip on or under the tongue (lingual or sublingual) or along the inside of the cheek (buccal cavity). As the thin film dissolves/erodes, drug is released and delivered to the blood stream either intragastrically, buccally or sublingually. Thin film intra-oral drug delivery technology offers advantages over other dosage forms, such as ingestible tablets, chewable tablets, orally dissolving tablets, soft gels, liquids or inhalants. These advantages include for example, (1) The potential to improve the onset of action, lower the dosing, and enhance the efficacy and safety profile of the medicament. Conversely, all tablet dosage forms, soft gels and liquid formulations primarily enter the blood stream via the gastrointestinal tract, which subjects the drug to degradation from stomach acid, bile, digestive enzymes and intestinal and/or hepatic first-pass effects. As a result, such formulations often require higher doses and generally have a delayed onset of action. Buccal and sublingual thin film drug delivery can avoid these issues and yield quicker onset of action at lower doses; (2) Improved dosing accuracy relative to liquid formulations since every strip is manufactured to contain a precise amount of the drug; (3) More accurate administration of drugs as well as improved compliance due to the intuitive nature of the dosage form and its inherent ease of administration. These properties are especially beneficial for pediatric, geriatric and neurodegenerative disease patients where proper and complete dosing can be difficult; and (4) Rapid dissolution without the need for water provides an alternative to patients with swallowing disorders and to patients suffering from nausea, such as those patients receiving chemotherapy.

Oral mucoadhesive films are typically applied to the tongue or adhere to the palate as soon as the subject closes his or her mouth. The palate is flat and is able to accommodate a larger size dosage form. The film can be designed such that it disintegrates and dissolves upon administration and the drug is released for oramucosal absorption.

In various embodiments, the present disclosure provides film compositions (alternatively referred to simply as films) comprising ketamine and a polymer. The film compositions are typically formulated to be suitable for use to deliver ketamine through oral transmucosal membrane, such as sublingual or buccal mucosa. As used herein, unless otherwise obvious from context, ketamine should be understood as including both ketamine base (the free base form) and a pharmaceutically acceptable salt thereof. However, in any of the embodiments described herein, the ketamine can exist substantially in its free base form. For example, in any of the embodiments described herein, the ketamine used for preparing the oral films herein can be substantially unprotonated (e.g., less than 10%, less than 5%, less than 2%, or less than 1%) prior to mixing with other ingredients of the oral films herein, see e.g., Examples 1-3. Without wishing to be bound by theories, it is believed that ketamine base is more transmucoally permeable compared to ketamine salts and thus can be advantageously incorporated into the film compositions herein. The ketamine is typically present in an amount of about 5% to about 30% by weight of the film composition. As used herein, unless otherwise obvious from context, the amounts of ketamine in a composition should be understood as the total amount of ketamine in the composition, regardless of its protonation state, expressed as the equivalent amount of ketamine base. Ketamine concentrations, permeation rate, and the like should be understood similarly, i.e., measured and/or calculated based on total ketamine (including base form, protonated form, complexed form, etc.) but with the final numeric value expressed as the equivalent value for ketamine base. Further, unless otherwise specified to the contrary, the weight percentages should be understood as based on dry weight.

The ketamine in the film compositions described herein is not limited to a particular enantiomer and can be in a racemic form, a substantially pure S-enantiomer (e.g., with less than 10% R-isomer, less than 5% R-isomer, less than 1% R-isomer, or less than 0.1% R-isomer), a substantially pure R-enantiomer (e.g., with less than 10% S-isomer, less than 5% S-isomer, less than 1% S-isomer, or less than 0.1% S-isomer), or a mixture of S- and R-isomers in any ratio. In some embodiments, the ketamine can be a substantially pure S-enantiomer (e.g., with less than 10% R-isomer, less than 5% R-isomer, less than 1% R-isomer, or less than 0.1% R-isomer). In some embodiments, the ketamine can be a substantially pure R-enantiomer (e.g., with less than 10% S-isomer, less than 5% S-isomer, less than 1% S-isomer, or less than 0.1% S-isomer). In some embodiments, the ketamine can be racemic.

S-ketamine was reported to be about 4-times more potent in NMDAR binding than R-ketamine. The in vivo potency in experimentally induced ischemic pain was also shown to be about 4-times different between the two enantiomers (Oye et al., J Pharmacol Exp Ther. March; 260(3):1209-13 (1992)). If, assuming the pharmacologic effect is 100% mediated by NMDAR and there are no differences in drug disposition systemically and locally, the dose to achieve the same receptor and pharmacologic effect is theoretically expected to be 1.6 times lower with S-ketamine than the racemic ketamine. A further study, by assuming the potency difference and pharmacokinetic exposure difference of about 2-fold accordingly, showed that the effect of the racemic mixture and the S-enantiomer of ketamine on temporal and spatial summation of pain were similar (Nielsen et al., British Journal of Anaesthesia, Br J Anaesth. 77(5):625-31 (1996)). Since there was no dose-response in Nielsen's study, it is not possible to conclude that the racemic form was 2-times less potent than S-ketamine. In addition, racemic ketamine prolonged the reaction time more than S-ketamine. Another study (Mathisen et al., Pain, 61(2):215-20 (1995)) suggested that S-ketamine may be 2-times more potent than racemic ketamine for orofacial pain. However, again, the study used fixed doses according to assumed differences in NMDAR binding. Evidence also suggested that ketamine has different potencies depending upon treatment of acute or chronic pain, and also different efficacy towards the same type of pain (such as neuropathic pain) from different causes (Niesters and Dahan, Expert Opin. Drug Metab. Toxicol., 8(11):1409-17 (2012)). More importantly, there has been conflicting evidence on whether the ketamine analgesic effect is mediated completely by NMDAR. For example, another NMDAR antagonist, MK-801, was shown to cause anti-analgesic effect in rodent species (Sigtermans et al., Pain, 145(3):304-11 (2009)). It was generally thought that the antidepressant effects of ketamine were mainly through the action of S-ketamine, because in vitro S-ketamine has about a 4-fold greater affinity than R-ketamine for NMDA receptor binding (Oye et al., J Pharmacol Exp Ther. March; 260(3):1209-13 (1992)). However, recent model animal studies have suggested that R-ketamine may be more effective as an antidepressant than S-ketamine (Yang et al., Transl Psychiatry, September 1; 5:e632 (2015)). In addition, R-ketamine was shown in animal models to be free of psychotomimetic side effects and abuse liability. Until now, the most abundant data on clinical antidepressant effects are largely reported from the use of racemic ketamine.

The polymer included in the films can be water-soluble, water-swellable, water-insoluble, or a combination of one or more either water-soluble, water-swellable or water-insoluble polymers. The polymer can include cellulose or a cellulose derivative. Specific examples of useful water-soluble polymers include, but are not limited to, polyethylene oxide, pullulan, hydroxypropylmethyl cellulose (HPMC), hydroxyethyl cellulose (HEC), hydroxypropyl cellulose (HPC), polyvinyl pyrrolidone (PVP), carboxymethyl cellulose (CMC), polyvinyl alcohol (PVA), sodium alginate, polyethylene glycol (PEO), xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water-insoluble polymers include, but are not limited to, ethyl cellulose (EC), hydroxypropyl ethyl cellulose (HPEC), cellulose acetate phthalate, hydroxypropyl methyl cellulose phthalate and combinations thereof.

As used herein, the phrase “water-soluble polymer” and variants thereof refer to a polymer that is at least partially soluble in water, and desirably fully or predominantly soluble in water, or absorbs water. Polymers that absorb water are often referred to as being water-swellable polymers. The materials useful with the present invention may be water-soluble or water-swellable at room temperature and other temperatures, such as temperatures exceeding room temperature. Moreover, the materials may be water-soluble or water-swellable at pressures less than atmospheric pressure. Desirably, the water-soluble polymers are water-soluble or water-swellable having at least 20 percent by weight water uptake. Water-swellable polymers having a 25 or greater percent by weight water uptake are also useful. In some embodiments, films formed from such water-soluble polymers may be sufficiently water-soluble to be dissolvable upon contact with bodily fluids such as saliva. The term “water-insoluble” refers to substances that are not dissolvable in water or upon contact with bodily fluids such as saliva.

Other polymers useful for incorporation into the films include biodegradable polymers, copolymers, block polymers and combinations thereof. It is understood that the term “biodegradable” is intended to include materials that chemically degrade, as opposed to materials that physically dissolve or erode (i.e., bioerodable materials). Among the known useful polymers or polymer classes which meet the above criteria are: poly(glycolic acid) (PGA), poly(lactic acid) (PLA), polydioxanes, polyoxalates, poly(a-esters), polyanhydrides, poly acetates, poly caprolactones, poly(orthoesters), poly amino acids, poly aminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific useful polymers include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers that provide mucoadhesive properties to the film, as well as a desired dissolution and/or disintegration rate. In some embodiments, one or more water-soluble polymers, as described herein, may be used to form the film. In other embodiments, however, it may be desirable to use combinations of water-soluble polymers and polymers that are water-swellable, water-insoluble and/or biodegradable, as provided herein. The inclusion of one or more polymers that are water-swellable, water-insoluble and/or biodegradable may provide films with slower dissolution or disintegration rates than films formed from water-soluble polymers alone. As such, the film may adhere to the mucosal tissue for longer periods or time, which may be desirable for delivery of ketamine in certain situations.

Desirably, the individual film has a small size, which is between about 0.5-1 inch by about 0.5-1 inch. Most preferably, the film is about 0.75 inches×0.5 inches or 1 inch×1 inch. The film should have good adhesion when placed in the buccal cavity or in the lingual or sublingual region of the user.

For instance, in some embodiments, the films may include polyethylene oxide alone or in combination with a second polymer component. The second polymer may be another water-soluble polymer, a water-swellable polymer, a water-insoluble polymer, a biodegradable polymer or any combination thereof. Suitable water-soluble polymers include, without limitation, any of those provided herein. In some embodiments, the water-soluble polymer may include hydrophilic cellulosic polymers, such as hydroxypropyl cellulose and/or hydroxypropylmethyl cellulose. In accordance with some embodiments, polyethylene oxide may range from about 20% to 100% by weight in the polymer component, more specifically about 30% to about 70% by weight, and even more specifically about 40% to about 60% by weight. In some embodiments, one or more water-swellable, water-insoluble and/or biodegradable polymers also may be included in the polyethylene oxide-based film. Any of the water-swellable, water-insoluble or biodegradable polymers provided herein may be employed. The second polymer component may be employed in amounts of about 0% to about 80% by weight in the polymer component, more specifically about 30% to about 70% by weight, and even more specifically about 40% to about 60% by weight.

The molecular weight of the polyethylene oxide also may be varied. In some embodiments, high molecular weight polyethylene oxide, such as about 4 million, may be desired to increase mucoadhesivity of the film. In some other embodiments, the molecular weight may range from about 100,000 to 900,000, more specifically from about 100,000 to 600,000, and even more specifically from about 100,000 to 300,000. In some embodiments, it may be desirable to combine high molecular weight (600,000 to 900,000) with low molecular weight (e.g., up to 300,000 (e.g., up to 200,000), such as from 100,000 to 300,000) polyethylene oxide in the polymer component. As used herein, when referring to polymers, unless otherwise specified or obvious contradictory to common practice, the molecular weight herein should be understood as the weight average molecular weight, M_{w}.

A variety of optional components and fillers also may be added to the films. These may include, without limitation: surfactants; plasticizers; polyalcohols; anti-foaming agents, such as silicone-containing compounds, which promote a smoother film surface by releasing oxygen from the film; thermo-setting gels such as pectin, carageenan, and gelatin, which help in maintaining the dispersion of components; inclusion compounds, such as cyclodextrins and caged molecules; coloring agents; and flavors.

Additives may be included in the films. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Film Designs

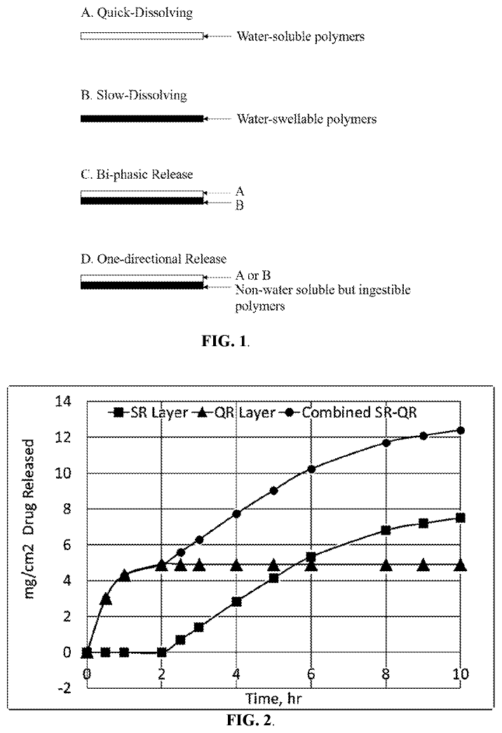

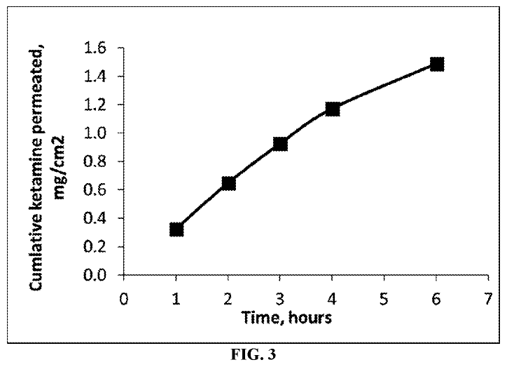

The oral films herein can have various designs. FIG. 1 shows, by example, four types of formulation: Quick-Releasing, Slow-Releasing, Bi-phasic Released, One-directional Release. Quick-Releasing (QR) Film is a fast release or quick release (QR) formulation which typically includes a water-soluble polymer, such as hydroxyl propyl methyl cellulose (HPMC), hydroxylpropyl cellulose (HPC), Povidone (PVP), polyvinyl alcohols (PVA), low molecular weight polyethylene oxide (PEO such as PolyOx N-10 supplied by Dow Chemical) starch-based polymers (Lycoat, manufactured by Roquette) etc. Slow-Releasing (SR) Film is a slow or sustained release (SR) formulation, which is prepared by using water-swellable polymers such as polycarboxylic acid (Carbopol), high molecular weight PEO, xanthan gum, chitosan etc. Bi-phasic Released Film can be prepared for example by combining (laminating) the QR and SR films into a single unit, which can result in a biphasic release profile (see e.g., an in vitro release profile shown in FIG. 2); the first being a QR to achieve high ketamine plasma concentrations quickly and the second being a SR to maintain longer efficacious ketamine plasma concentrations. One-directional Release Film, a dual-layer design, typically is a combination of a QR film or a SR film, and a water-insoluble layer using ingestible polymers to prevent drug in the QR film or SR film from flowing into the mouth cavity and intragastric tract. Water-insoluble but ingestible polymers can include hydrophilic (but non-water-soluble) polymers such ethyl cellulose (e.g. Ethocel by Dow Chemical), Eudragit (polymethacrylates manufacture by Evonik) etc.

Typically, the ketamine is included in the mucoadhesive layer of the film herein (QR or SR film). However, in some embodiments, additional ketamine can be included in a reservoir layer, for example, sandwiched between a SR film and a barrier layer, or between two SR films.

Other film designs are also suitable and would be apparent to those skilled in the art in view of the present disclosure. Additionally, the films herein can be combined in a variety of ways, for example, in a package according to a desired need.

Quick-Release Film Composition

Certain embodiments of the present invention are directed to novel quick release film compositions. The quick-release (QR) films herein typically include ketamine dissolved or dispersed in a water-soluble polymer matrix. In some embodiments, the ketamine in the QR film can be substantially in its free base form. In some embodiments, ketamine base can be used as the active ingredient for preparing the QR film. For example, in some embodiments, the ketamine used for preparing the QR films herein can be substantially unprotonated (e.g., less than 10%, less than 5%, less than 2%, or less than 1%) prior to mixing with other ingredients of the QR films herein, see e.g., Example 1. The ketamine is typically present in an amount of about 5% to about 30% by weight of the QR film.

In some embodiments, the QR film further comprises a permeation enhancer, a plasticizer, an antioxidant, or any combination thereof. For example, in some embodiments, the QR film comprises (a) ketamine, (b) a water-soluble polymer; and (c) a permeation enhancer. In some embodiments, the QR film further comprises (d) an antioxidant. In some embodiments, the QR film further comprises (e) a plasticizer. In some embodiments, the QR film comprises all of (a)-(e). However, in some embodiments, the QR film can also be free or substantially free of a permeation enhancer. In some embodiments, the QR film can also be free or substantially free of an antioxidant. In some embodiments, the QR film can also be free or substantially free of a plasticizer. The QR film can also optionally include one or more ingredients typically included in an oral film composition, such as taste-masking agents (e.g., flavors, sweeteners, flavor enhancers etc.), antifoaming agents, surfactants, emulsifying agents, thickening agents, binding agents, cooling agents, saliva-stimulating agents, antimicrobial agents, and combinations thereof.

Various water-soluble polymers are suitable for the QR films herein. Suitable water soluble polymers include any of those described herein. Typically, such water-soluble polymers are edible. In some embodiments, the water-soluble polymer can be synthetic or partially synthetic polymers, or naturally occurring biopolymers that can form films and are water-soluble, for example, cellulose derivatives, polyvinylalcohol, polyacrylates and polyvinylpyrrlidone. Non-limiting useful cellulose derivatives include hydroxypropylmethylcellulose, carboxymethylcellulose, sodium carboxymethylcellulose, hydroxyethylcellulose, hydroxypropylcellulose, methylcellulose, and hydroxypropylmethylcellulose. Water-soluble polysaccharides derived from plants or microbes, such as pullulan, trantan, alginate, dextrin and pectins, can also be used. Non-limiting useful water-soluble polymers also include proteins such as gelatin or other gel-forming proteins, starch and starch derivatives, gelatin, polyvinylpyrrilidone, gum Arabic, pullulan, acrylates, polyethylene oxide (e.g., polyox 10, polyox 80, polyox 205, polyox 301, polyox 750) or copolymers of methylvinylether and maleic acid anhydride. In some embodiments, the QR film comprises one or more water-soluble polymers selected from hydroxyl propyl methyl cellulose (HPMC), hydroxylpropyl cellulose (HPC), Povidone, polyvinyl alcohols (PVA), low molecular weight polyethylene oxide (PEO such as PolyOx N10 supplied by Dow Chemical), and starch-based polymers (Lycoat, manufactured by Roquette). The water-soluble polymer(s) typically are present in an amount of about 30% to about 90% by weight of the QR films (dry weight), for example, about 30%, about 40%, about 50%, about 60%, about 70%, about 80%, about 90%, or any ranges between the specified values, by weight of the QR film. In some embodiments, the QR film comprises the water-soluble polymer in the amount ranging from about 40% to about 80%, about 50% to about 80%, or about 50% to about 70% by weight.

As those skilled in the art would understand, some agents can have dual or multiple functions. For clarity of calculation of weight percentages, as used herein, when a dual (or multiple) functional agent is included in a film herein, the amount of that agent is proper as long as it can fit into one of the specified weight limits according to one of its functions, unless otherwise obvious from context. It should be clear that the total amount of ingredients should not exceed 100% and the same agent is calculated only once towards the total amount.

In some embodiments, the QR film can include one or more permeation enhancers, which can improve ketamine oral transmucosal permeation efficacy. Suitable permeation enhancers include propandiol, dexpanthenol, saturated or unsaturated fatty acids or esters (e.g., oleic acid), hydrocarbons, linear or branched fatty alcohols (e.g., oleic alcohol), dimethylsulfoxide, propylene glycol, decanol, dodecanol, 2-octyldodecanol, glycerine, ethanol or other alcohols. In some embodiments, the QR film can comprise oleic alcohol as a permeation enhancer. In some embodiments, the QR film can comprise one or more permeation enhancers selected from dimethyl sulfoxide (DMSO), oleic alcohol, oleic acid, oleyl oleate, levulinic acid, propylene glycol, dipropylene glycol, ethanol, and surfactants. In some embodiments, the QR film can also be free or substantially free of a permeation enhancer. For example, in some embodiments, the QR film can be free or substantially free of oleic alcohol and oleic acid. The permeation enhancer, when present, is typically included in the amount of about 1% to about 35% by weight of the QR film (dry weight), for example, about 2%, about 5%, about 10%, about 15%, about 20%, about 25%, about 30%, about 35%, or any ranges between the specified values, by weight of the QR film. For example, in some embodiments, the permeation enhancer is present in an amount of about 5% to about 30% or about 2% to about 20% by weight of the QR film.

Other ingredients can also be optionally included in the QR film described herein. For example, in some embodiments, the QR film can comprise an antioxidant (e.g., tocopherol or derivatives, such as tocopherol acetate). Antioxidant(s), when present, typically are present in an amount effective to reduce the amount of oxidative degradation of ketamine compared to a substantially same film without the antioxidant(s). The amount of antioxidants can vary, for example, can range from about 0.1% to about 5% (e.g., about 0.1%, about 0.3%, about 0.5%, about 0.7%, about 1%, about 2%, about 5%, or any ranges between the recited values) by weight of the QR film, for example, ranging from about 0.1% to about 1% by weight of the QR film. In some embodiments, the QR film can comprise an antioxidant in an amount ranging from about 0.2% to about 1% by weight.

In some embodiments, the QR film can comprise a plasticizer. Non-limiting useful plasticizers include polyalkylene oxides, such as polyethylene glycols (e.g., PEG400), polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like. Plasticizers, when present, typically are present in an amount ranging from about 0.5% to about 30% by weight of the QR film, for example, ranging from about 0.5% to about 20% or about 5% to about 15% by weight of the QR film.

In some specific embodiments, the QR films can have the following ingredients:

| | Typical | Preferred | Specific |
|---|---|---|---|
| Ingredient | Amount | amount | amount |
| Water soluble polymer (e.g., | 30-90% | 40-80% or | 54% |
| HPMC, PolyOx N-10l Kollidon | | 50-70% | |
| 90F) | | | |
| Release-modifying polymer (e.g., | 0-30% | 5-25% or | 0% or |
| Eudragit, EC, Kollidon VA64) | | 10-20% | 13% |
| Active ingredient (e.g., Ketamine | 5-30% | 10-25% | 15% |
| Free Base) | | | |
| Antioxidant (e.g., Tocopherol | 0-5% | 0.1-1% or | 0% or |
| acetate, citric acid) | | 0.2-1% | 0.5% |
| Plasticizer (e.g., PEG 400 and/or | 0-30% | 0.5-20% or | 0% or |
| propylene glycol) | | 5-15% | 7.5% |
| Permeation enhancer (e.g., Oleic | 0-35% | 5-30% or | 0% or |
| alcohol, dipropylene glycol, | | 2-20% | 10.0% |
| Transcutol P) | | | |
| Total | 100% | 100% | 100% |

All weight percentages in the table above are based on dry weight, and should be understood as preceded by the word “about”. The ingredients are exemplary in the table and other suitable ingredients are exemplified herein.

The QR film herein can also be characterized by its dissolution profile. For example, in some embodiments, the QR film is characterized in that it releases substantially all of the ketamine therein in less than 30 minutes (e.g., about 10 minutes or less, about 5 minutes or less), when tested in vitro in a dissolution test using USP Apparatus 2. In some embodiments, the QR film can also be characterized in that it is dissolved and/or disintegrated in about 10 minutes or less, about 5 minutes or less (about 1 minute or less) after administered lingually, sublingually or buccally, to a user.

The dissolution of the QR film herein can be adjusted, for example, by including in the film a modified polymer as a release-modifying polymer. For example, in some embodiments, the QR film herein can include polyvinylpyrrlidone (PVP, e.g., Kolidon 90F) in combination of a copolymer of polyvinylpyrrlidone and vinyl acetate (e.g., Kolidone VA64). In some embodiments, a ratio of PVP to copolymer of PVP-vinyl acetate can range from about 10:1 to about 2:1 by weight. For example, in some embodiments, the QR film can include a combination of PVP and copolymer of PVP-vinyl acetate, with the ratio of PVP to copolymer of PVP-vinyl acetate of about 4:1 by weight.

The QR film herein can be used as a separate film, either alone or in combination with another therapy, such as another film described herein (e.g., a SR film), or can be laminated with another film (e.g., a SR film herein) to form a bilayer film composition.

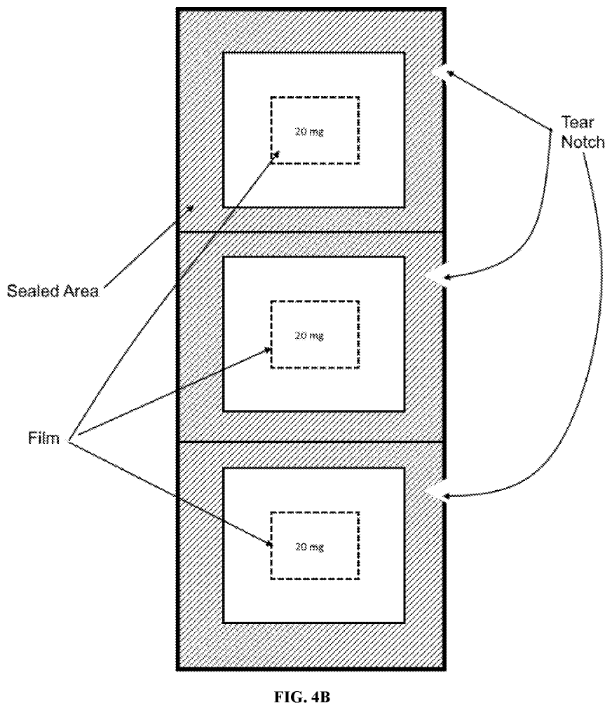

As the QR film herein can be dissolved and/or disintegrated rapidly, multiple applications of QR films are possible. In some embodiments, the present disclosure also provides a kit comprising multiple films (e.g., QR films described herein), which can allow a user to conveniently apply more than one QR films, which can be the same or different, in a relatively short period of time. For example, as shown in FIGS. 4A and 4B, a pouch can include two or three films, e.g., two or three QR films, each containing about 20 mg ketamine. The films can be sealed by any suitable material, and a tear notch can be used by a user to conveniently retrieve each film from the pouch when needed.

For example, the oral films (e.g., QR films) herein can be applied on the tongue. When the user closes his/her mouth, the oral film is immediately transferred to the upper platelet and stay there till it completely dissolves. After the transfer, the tongue now is available for the next oral film. After the application and transfer of the 2^{nd} film, the tongue can be dosed with the 3^{rd} film, and so on . . . . Therefore, in some embodiments, many oral films such as QR films herein can be successively applied, one after another in a short time, to provide enough total dosage of drug. These films can be taken conveniently from exemplary pouch constructions as shown in FIGS. 4A and 4B.

Slow-Release Film Composition

Certain embodiments of the present invention are directed to novel slow release (SR) film compositions. The slow-release (SR) films herein typically include ketamine in a mucoadhesive polymer. Mucoadhesive polymers are known, for example, as described in Kavitha et al., “Novel Mucoadhesive Polymers—A review,” Journal of Applied Pharmaceutical Science 01(08): 37-42 (2011). In some embodiments, the SR film comprises a therapeutically effective amount of ketamine dissolved or dispersed in a mucoadhesive polymer matrix. In some embodiments, the ketamine in the SR film can be substantially in its free base form. In some embodiments, ketamine base can be used as the active ingredient for preparing the SR film. For example, in some embodiments, the ketamine used for preparing the SR films herein can be substantially unprotonated (e.g., less than 10%, less than 5%, less than 2%, or less than 1%) prior to mixing with other ingredients of the SR films herein, see e.g., Example 2. The ketamine is typically present in an amount of about 5% to about 30% (e.g., about 5%, about 10%, about 15%, about 20%, about 25%, about 30%, or any range between the recited values) by weight of the SR film.

In some embodiments, the SR film further comprises a permeation enhancer, a plasticizer, an antioxidant, or any combination thereof. For example, in some embodiments, the SR film comprises (a) ketamine, (b) a mucoadhesive polymer (e.g., water-swellable polymer); and (c) a permeation enhancer. In some embodiments, the SR film further comprises (d) an antioxidant. In some embodiments, the SR film further comprises (e) a plasticizer. In some embodiments, the SR film comprises all of (a)-(e). However, in some embodiments, the SR film can also be free or substantially free of a permeation enhancer. In some embodiments, the SR film can also be free or substantially free of an antioxidant. In some embodiments, the SR film can also be free or substantially free of a plasticizer. The SR film can also optionally include one or more ingredients typically included in an oral film composition, such as taste-masking agents (e.g., flavors, sweeteners, flavor enhancers etc.), antifoaming agents, surfactants, emulsifying agents, thickening agents, binding agents, cooling agents, saliva-stimulating agents, antimicrobial agents, and combinations thereof.

Various polymers can be used as a mucoadhesive polymer for the SR films herein. Typically, the mucoadhesive polymer is a water swellable polymer. In some embodiments, the mucoadhesive polymer comprises one or more polymers selected from cellulose derivatives, polyacrylic acids, polyacrylates, polyethylene oxides, polyvinyl pyrrolidones, polyvinyl alcohols, tragacanth, alginates, gum (including karaya gum, guar gum, xanthan gum), soluble starch, gelatin, lectin, pectin, and chitosan. In some embodiments, the mucoadhesive polymer comprises one or more polymers selected from a hydrophilic polymer, a polysaccharide and its derivatives, and a hydrogel. In some embodiments, the mucoadhesive polymer comprises one or more polymers selected from polyacrylic acids, polyacrylates, celluloses, e.g., carboxy celluloses (e.g., sodium carboxymethyl cellulose), hydroxyalkyl cellulose (e.g., hydroxypropylcellulose, hydroxyethylcellulose and hydroxyethyl ethyl cellulose)), polyvinylpyrrolidone, and polyvinyl alcohol. In some embodiments, the mucoadhesive polymer comprises one or more polymers selected from Carbopol (polyacrylic acid), carboxymethyl cellulose, carboxyethyl cellulose, hydroxypropyl cellulose, hydroxypropylmethyl cellulose, and gum.

In some embodiments, the mucoadhesive polymer can comprise two or more water swellable polymers herein. For example, in some embodiments, the water swellable polymers can include a polyacrylic acid (e.g., Carbopol) and a carboxy cellulose (e.g., sodium carboxymethyl cellulose). Without wishing to be bound by theories, it is believed that the polyacrylic acid (e.g., Carbopol) can provide strong adhesion by ionic interaction when wetted with saliva on oral mucosal tissues.

The mucoadhesive polymers typically are present in an amount of about 15% to about 80% by weight of the SR films (dry weight), for example, about 15%, about 20%, about 30%, about 40%, about 50%, about 60%, about 70%, about 80%, or any ranges between the specified values, by weight of the SR film. In some embodiments, the SR film comprises the mucoadhesive polymer in the amount ranging from about 15% to about 60% by weight.

In some embodiments, the SR film can also include one or more permeation enhancers, which can improve ketamine oral transmucosal permeation efficacy. Suitable permeation enhancers include propandiol, dexpanthenol, saturated or unsaturated fatty acids or esters (e.g., oleic acid), hydrocarbons, linear or branched fatty alcohols (e.g., oleic alcohol), dimethylsulfoxide, propylene glycol, decanol, dodecanol, 2-octyldodecanol, glycerine, ethanol or other alcohols. In some embodiments, the SR film can comprise oleic acid as a permeation enhancer. In some embodiments, the SR film can comprise one or more permeation enhancers selected from dimethyl sulfoxide (DMSO), oleic alcohol, oleic acid, oleyl oleate, levulinic acid, propylene glycol, dipropylene glycol, ethanol, and surfactants. In some embodiments, the SR film can also be free or substantially free of a permeation enhancer. For example, in some embodiments, the SR film can be free or substantially free of oleic alcohol and oleic acid. The permeation enhancer, when present, is typically included in the amount of about 1% to about 35% by weight of the SR film (dry weight), for example, about 2%, about 5%, about 10%, about 15%, about 20%, about 25%, about 30%, about 35%, or any ranges between the specified values, by weight of the SR film. For example, in some embodiments, the permeation enhancer is present in an amount of about 1% to about 30%, about 5% to about 30%, or about 2% to about 20%, by weight of the SR film.

Other ingredients can also be optionally included in the SR film described herein. For example, in some embodiments, the SR film can comprise an antioxidant (e.g., tocopherol or derivatives, such as tocopherol acetate). Antioxidant(s), when present, typically are present in an amount effective to reduce the amount of oxidative degradation of ketamine compared to a substantially same film without the antioxidant(s). The amount of antioxidants can vary, for example, can range from about 0.1% to about 5% (e.g., about 0.1%, about 0.3%, about 0.5%, about 0.7%, about 1%, about 2%, about 5%, or any ranges between the recited values) by weight of the SR film, for example, ranging from about 0.1% to about 1% by weight of the SR film. In some embodiments, the SR film can comprise an antioxidant in an amount ranging from about 0.2% to about 1% by weight.

In some embodiments, the SR film can comprise a plasticizer. Non-limiting useful plasticizers include polyalkylene oxides, such as polyethylene glycols (e.g., PEG400), polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like. Plasticizers, when present, typically are present in an amount ranging from about 0.5% to about 50% by weight of the SR film, for example, ranging from about 0.5% to about 20%, about 5% to about 45%, or about 10% to about 40%, by weight of the SR film.

In some specific embodiments, the SR films can have the following ingredients:

| | Typical | Preferred | Specific |
|---|---|---|---|
| Ingredient | Amount | amount | amount |
| Water swellable polymer (e.g., Na | 15-80% | 15-60% or | 45% |
| CMC Type 12, Carbopol 971P | | 30-60% | |
| and/or HPMC E5) | | | |
| Active ingredient (e.g., Ketamine | 5-30% | 10-25% | 16% |
| Free Base) | | | |
| Antioxidant (e.g., Tocopherol | 0-5% | 0.1-1% or | 0% or |
| acetate) | | 0.2-1% | 0.5% |
| Plasticizer (e.g., PEG 400 and/or | 0-50% | 5-45% or | 0% or |
| propylene glycol) | | 10-40% | 32% |
| Permeation enhancer (e.g., Oleic | 0-35% | 1-30% or | 0% or |
| acid) | | 2-20% | 5.0% |
| Other ingredients (e.g., sweetener | 0-5% | 0.1-1% or | 0.6% |
| such as sucralose) | | 0.2-1% | |
| Total | 100% | 100% | 100% |

All weight percentages in the table above are based on dry weight, and should be understood as preceded by the word “about”. The ingredients are exemplary in the table and other suitable ingredients are exemplified herein.

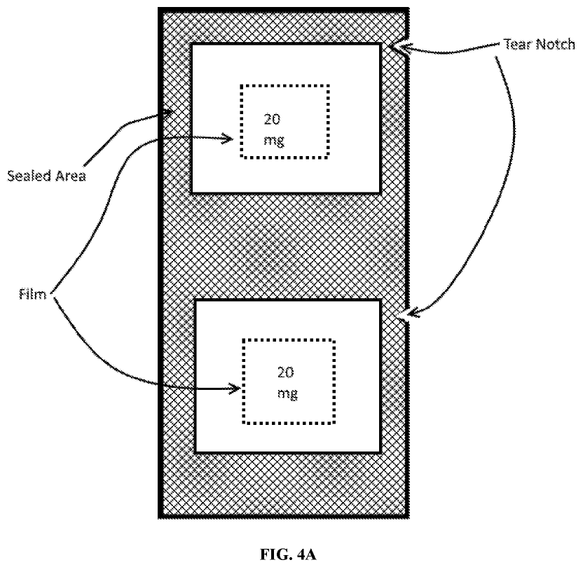

The SR film herein can also be characterized by its dissolution profile. For example, in some embodiments, the SR film is characterized in that, when tested in vitro using cultured human buccal tissue in a Franz model, the film composition delivers ketamine through the human buccal tissue at a rate of at least about 0.1 mg/cm²*h, for example, about 0.1 mg/cm²*h to about 2 mg/cm²*h, or about 0.1 mg/cm²*h to about 5 mg/cm²*h, for example, about 0.1 mg/cm²*h, 0.2 mg/cm²*h, about 0.5 mg/cm²*h, about 1 mg/cm²*h, about 1.5 mg/cm²*h, about 2 mg/cm²*h, about 5 mg/cm²*h, or any ranges between the recited values for the first hour, first two hours, or first 4 hours. As used herein, the unit mg/cm²*h and its variants such as mg/cm²/h or mg/h/cm² should be understood as mg per cm² per hour. In some embodiments, when specifically referenced as measured by an in vitro method using cultured human buccal tissue in a Franz model, the ketamine permeation rate is measured in accordance with the method described in Example 2 of this disclosure. For example, for a substantially same film as in Example 2, the testing method should also yield substantially the same ketamine release rate as shown in Example 2 (FIG. 3), within experimental errors accepted by those skilled in the art. Further, as used herein, the rate of ketamine permeation through the buccal tissue in vitro should be understood as the average rate observed for a specified testing period, for example, in the first hour, first two hours, or first four hours.

The SR film herein can be used as a separate film, either alone or in combination with another therapy, such as another film described herein (e.g., a QR film), or can be laminated with one or more other films (e.g., a QR film herein) to form a bilayer or multilayer film composition.

As the SR film herein can provide a sustained release of ketamine, in some embodiments, it can be used to provide a maintenance dose of ketamine in conjunction with an immediate release or quick release ketamine delivery composition and/or device. For example, in some embodiments, the present disclosure also provides a kit comprising a QR film and a SR film described herein, which can allow a user to conveniently apply a QR film as an initial dose of ketamine and then a SR film as a maintenance dose. The QR film, when applied, e.g., sublingually, can achieve a fast absorption of ketamine and rapid on-set of an intended pharmacologic action, and the SR film, when applied, e.g., via the buccal site, can achieve a relatively long and sustained release of ketamine to maintain a longer therapeutic effect. Various combinations of QR and SR films are possible, depending on the desired pharmacological effects. For example, as shown in FIGS. 4A and 4B, a pouch can include two or three films, e.g., one QR and one SR film, or two QR and one SR film, each containing about 20 mg ketamine. Similarly, the films can be sealed by any suitable material, and a tear notch can be used by a user to conveniently retrieve each film from the pouch when needed. Other packaging systems are also possible and the amount of ketamine in the QR and SR films can be adjusted according to the desired pharmacological effect. In some embodiments, the amount of ketamine in the QR film is the same or different from that in the SR film.

Bi-Phasic Release

In some cases, a biphasic delivery of ketamine can be advantageous. The biphasic delivery can result in a biphasic pharmacokinetic profile, which can provide a fast on-set and sustained effect, e.g., analgesia or antidepressant effect. For example, the acute pain arising from wounds from battlefield or an accident needs a fast on-set of analgesia, thus rapid ketamine absorption is advantageous. As ketamine has a short half-life (˜2-3 hours), maintenance of pain management over hours or days needs sustained delivery and exposure of ketamine following the initial fast absorption phase.

The biphasic delivery of ketamine can be achieved in various ways. For example, in some embodiments, a quick release dosage form (e.g., a QR film herein) can be applied first, e.g., to the sublingual mucosa, to achieve a quick delivery of ketamine, which can be followed by applying a sustained release dosage form, such as a SR film herein, e.g., to the buccal mucosa, to achieve a sustained delivery of ketamine.

In some embodiments, a Bi-phasic Released Film can be prepared by combining the QR and SR films into a single unit, which results in a two-phase in vitro release profiles (e.g., FIG. 2); the first being a QR to achieve high concentrations quickly and the second being a SR to maintain longer efficacious concentrations. For example, in some embodiments, the QR film and SR film can be laminated as a unit dosage form to achieve a biphasic delivery of ketamine. In some embodiments, the present disclosure provides a bilayer film with a QR film (e.g., any of those described herein) and SR film (e.g., any of those described herein) laminated together. Typically, the bilayer film is configured such that the QR film layer is in contact with oral cavities of a user initially. In other words, the QR film layer is the “outer” layer and the SR film layer is the “inner” layer. FIG. 1(C) shows an example of such bilayer film. Without wishing to be bound by theories, when the bilayer film is applied to oral mucosal tissues, the QR film can rapidly release ketamine for absorption through the oral mucosal tissues and therefore provide fast ketamine exposure and a fast on-set of effects, e.g., analgesia and/or antidepressant effects. The SR film, being a slow-dissolving and sustained formulation, can then provide a sustained release of ketamine, for example, to maintain the pharmacological effects, such as analgesic or antidepressant effect for a prolonged time. In some embodiments, the bilayer film can be characterized in that when tested in an in vitro release test, it releases ketamine at a rate of at least about 0.5 mg per cm² per hour (0.5 mg/cm²*h) (e.g., about 1 mg/cm²*h to about 20 mg/cm²*h, or about 1 mg/cm²*h to about 15 mg/cm²*h) for the first hour or first two hours, and releases ketamine at a rate of about 0.5 mg/cm²*h to about 5 mg/cm²*h thereafter, e.g., for an extended period of about 2 hours, about 4 hours, about 6 hours, or about 8 hours or longer. The rate of ketamine release in this in vitro method should be understood as the average rate observed for the relevant testing period.

By choosing different QR films and SR films, the rate and duration of delivery of ketamine can be controlled, either as separate films or as a bilayer film. An example of in vitro dissolution of a bilayer film is shown in FIG. 2, which is configured to deliver about 5 mg/cm² of ketamine in the first two hours through the QR film (QR layer) and about 7 mg/cm² of ketamine from hour 3 to hour 10. Although FIG. 2 shows an in vitro dissolution of a bilayer film, similar dissolution profile can be achieved by applying two separate films that are substantially similar to the QR and SR films in the bilayer film, respectively. The rate of release can also be adjusted, for example, to achieve a delivery of about 2 mg to about 60 mg (e.g., about 3 mg, about 5 mg, about 15 mg, about 30 mg, about 60 mg, or any ranges between the specified values) of ketamine in the first hour or first 2 hours and followed by about 2 mg to about 50 mg (e.g., about 5 mg, about 15 mg, about 25 mg, about 50 mg, or any ranges between the specified values) of ketamine thereafter for an extended period of about 2 hours, about 4 hours, about 6 hours, or about 8 hours, or longer.

One-Directional Film Composition

In some embodiments, a diffusion barrier layer is also included for the films (e.g., QR, SR, or bilayer films) described herein. The diffusion barrier layer can prevent or restrict loss of active ingredients to the oral cavity and ultimately to the gastrointestinal tract.

As shown in FIG. 1(D), either a QR film or a SR film can be laminated with a diffusion barrier layer, such that the delivery of ketamine is one-directional. In addition, the bi-layer film described herein (e.g., FIG. 1(c)) can also be laminated with a diffusion barrier layer. (Not shown in FIG. 1). In such embodiments, it is preferably the SR film is laminated directly with the diffusion barrier layer. Thus, when applied, the QR film is first in contact with the oral mucosal tissues, and after the QR film is dissolved/disintegrated, the SR film adheres to the oral mucosal tissues and provides a sustained release of ketamine through the oral mucosal tissues.

Any such barrier layers known in the art can be used herein. Typically, the barrier layer comprises a water-insoluble but ingestible polymer. Any of the water-insoluble polymer can be used. For example, in some embodiments, the water-insoluble but ingestible polymer can comprise a cellulose (e.g., ethyl cellulose), a polyacrylate (e.g., polymethylacrylate), or a combination thereof.

Various methods can be used for preparing the films herein. Typically, the films herein can be produced by wet casting or deposition followed by drying. For example, the ingredients can be combined with a solvent, which can be water, a polar organic solvent including, but not limited to, ethanol, isopropanol, acetone, or any combination thereof. The wet composition can then be casted and dried to provide the film herein. It should be noted that the wet composition by itself is also a novel composition.

The films herein typically include only ketamine as the active ingredient. However, in some embodiments, the film can also include one or more additional active ingredients. In some embodiments, the film can also be used in combination with one or more additional active ingredients, whether present in the film or in a separate dosage form. For example, in some embodiments, the film compositions herein can be used in combination with co-medications that can reduce side effects of ketamine or enhance ketamine therapeutic effects. Non-limiting useful medications that can be included as co-medications include those that can reduce risk in hypertension induced by relatively high and transient concentration of ketamine, such as clonidine. Non-limiting useful medications that can be included as co-medications also include those that can enhance the antidepressant efficacy, such as scopolamine. Non-limiting useful medications that can be included as co-medications also include those that can enhance the management of pain, and/or reduce the addiction and abuse potential by lowering consumptions of such medications, such as opioids. In some embodiments, the co-medications can be included with ketamine in a single film or in separate dosage forms (e.g., separate films). When in separate dosage forms, the co-medications can also be applied/administered to a subject user concurrently or at different times.

Method of Administering Ketamine

The films herein provide alternative options for administering ketamine to a subject in need thereof. As exemplified herein, ketamine has been tested in clinical trials for various diseases or disorders. In some embodiments, the present disclosure also provides a method of treating a disease or disorder where administering ketamine can be beneficial. In such embodiments, the method can comprise administering one or more films herein. The films herein are typically administered (or applied) to an oral mucosal membrane. For example, in some embodiments, the films herein can be administered on or under the tongue (lingual or sublingual). In some embodiments, the film can be administered along the inside of the cheek (buccal cavity).

Nonlimiting diseases or disorders suitable to be treated by ketamine include any of those known in the art, for example, depression or pain. Applications of ketamine for pain management have been reported, which include for example: (1) The management of acute perioperative pain, such as reviewed by (Jouguelet-Lacoste, et al., Pain Medicine, 16(2):383-403 2015), which included clinical trials representing 2,482 patients, 1,403 of whom received ketamine, demonstrating that low-dose intravenous (IV) ketamine reduces opioid consumption by 40% and lowers pain scores. No major complications have been reported with low-dose IV infusion of ketamine when given up to 48 hours after surgery. Overall, it appears that, when used in a low-dose range (IV infusion rate less than 1.2 mg/kg/h), a continuous ketamine infusion is not associated with serious side effects. Only one study out of the 25 reported serious adverse events (excessive bleeding in both the placebo and ketamine groups and an anaphylactic reaction in the ketamine group). Out of the 10 studies using only an intraoperative infusion of ketamine that reported on safety, four reported no psychotomimetic events, while the remaining six reported no difference in the incidence of those events between ketamine and placebo. Out of the seven studies assessing a 24-hour postoperative IV infusion, one reported no psychotomimetic events and five reported no differences between the ketamine and control groups. The last of these studies reported a positive correlation between the incidence of vivid dreams and the dose of ketamine used (5 mg/h, 10 mg/h, and 20 mg/h). Out of the eight studies that assessed a 48-hour postoperative infusion, five reported no psychotomimetic events, while two reported an equal incidence in both ketamine and placebo groups regarding hallucinations and unpleasant dreams. In all of the 39 clinical trials included in our review, which assessed low-dose ketamine (IV infusion rate of less than 1.2 mg/kg/h and bolus dose less than 1 mg/kg), there were no indications of liver toxicity. Reports of liver toxicity have only been observed in abusers of ketamine or in chronic pain patients who received repeated and long-term doses of ketamine (two continuous IV 100-hour infusions of ketamine at an infusion rate of 10-20 mg/h). (2) Long-term infusion of ketamine for treatment of chronic pain, including neuropathic pains, cancer pain, CRPS, etc. Sixty patients with severe complex regional pain syndrome (CRPS-1) participated in a double-blind randomized placebo-controlled parallel-group trial (Sigtermans et al., Pain, 145(3):304-11 (2009)), where patients were given a 4.2-day intravenous infusion of low-dose ketamine using an individualized stepwise tailoring of dosage based on effect (pain relief) and side effects (nausea/vomiting/psychotomimetic effects), which resulted in significant pain relief without functional improvement. Treatment with ketamine was safe with psychotomimetic side effects that were acceptable to most patients. In another study (Harbut and Correll, Pain Medicine, 3(2):147-55 (2002)), one patient was carefully titrated with slowly increasing doses of ketamine up to that level which just began to make her feel mildly inebriated; visual analog scale (VAS) reduction correlated well with the pharmacokinetic profile. The patient did not experience hallucinations, dysphoria, or sedation. The patient did experience a period of mild inebriation that started several hours after her infusion began, lasted for 2-3 days, but then resolved even though her infusion continued. The patient otherwise did well with no other observable side effects. In an additional study (Correll and Graham, Pain Medicine, 7(1):92-5 (2006)), a total of 33 patients with a diagnosis of CRPS underwent ketamine treatment with ketamine infusion rates of about 23 mg/hr for about 4.7 days. Mean immediate response (% relief) was as high as 92%. The most frequent side effect observed in patients receiving this treatment was a feeling of inebriation. Hallucinations occurred in six patients. Less frequent side effects also included complaints of lightheadedness, dizziness, and nausea. In four patients, an alteration in hepatic enzyme profile was noted; the infusion was terminated and the abnormality resolved following discontinuation of the ketamine infusion. There were no suicidal and/or homicidal tendencies. No ketamine related addictive behaviors were observed. (3) Reduction of postoperative pain as well as reduced opioid consumption was noted. One meta-analysis showed that IV ketamine infusion at sub-anesthetic doses significantly reduced postoperative pain scores after laparoscopic cholecystectomy (LC). Significant differences were found regarding opioid consumption at 12, 24 and 48 h after LC. In addition, there were fewer adverse effects in the ketamine groups (Zhu, et al., International Journal of Surgery, 49:1-9 (2018)). A similar meta-analysis with 14 RCTs comprising 649 patients also showed that supplemental perioperative ketamine reduced postoperative opioid consumption up to 24-hours following spine surgery. The ketamine group also reported lower postoperative pain scores at 6, 12, and 24 hours (Pendi et al., Spine, 1;43(5):E299-E307 (2018)). One hundred and fifty patients received an S-ketamine bolus dose of 0.5 mg/kg and an infusion 0.25 mg/kg/hr or placebo. A mean difference of 42 mg in the morphine consumption 0 to 24 hours postoperatively was observed. There were no significant differences regarding acute pain, nausea, vomiting, hallucinations, or nightmares. Back pain at 6 months postoperatively compared with preoperative pain was significantly better improved in the ketamine group compared with the placebo group. (4) Ketamine has also been used in emergency departments (ED) for acute pain control. Literature review of eight randomized controlled trials showed that subdissociative-doses (usually less than 1 mg/kg) were effective and safe to use alone or in combination with opioid analgesics for the treatment of acute pain in the ED (Motov et al., Journal of Emergency Medicine, 51(6):752-757 (2016)). Motov and his colleagues evaluated the analgesic efficacy and safety of subdissociative intravenous-doses of ketamine in the ED patients in a prospective, randomized, double-blind trial, where 45 patients received ketamine at 0.3 mg/kg or morphine at 0.1 mg/kg by intravenous push during 3 to 5 minutes. There were no significant differences in the primary change in mean pain scores and in the changes in clinical vital signs between the ketamine and morphine groups (Motov et al, Annals of Emergency Medicine, 66(3):222-229 (2015)).

Major depressive disorder (MDD) affects about 20 percent of the population sometime during their lives (Fava and Kendler, Neuron, 28(2):335-41 (2000)). However, relevant scientific efforts and the pharmaceutical R&D pipeline have not produced major advancements in antidepressant treatment modalities during the past half-century (Otte et al., Nat Rev Dis Primers, 15;2:16065 (2016)). For example, all current major antidepressant drugs share the same monoamine molecular targets in the brain as their prototypes from the 1950s and share similar issues, including moderate therapeutic effects, delayed onset of efficacy, short-term and long-term side-effects leading to poor treatment adherence, and significant relapse risk despite continued long-term treatment (Trivedi et al., Am J Psychiatry, 163(1):28-40 (2006)). Recently, clinical and basic biology data have been accumulating to indicate that ketamine can be an effective antidepressant with consistent, rapid and sustained antidepressant effects at low doses (Berman et al., Bio Psychiatry, 15;47(4):351-4 (2000)). Unlike conventional antidepressants, which generally take weeks to months to take full effect, ketamine lifts depression in as little as a few hours, and such effects may extend to days after a single subanesthetic-intravenous infusion (Schwartz et al., Evid Based Ment Health, 19(2):35-8 (2016)). Ketamine has been widely used in off-label treatment for MDD, mostly with short-term intravenous infusions (Andrade, J Clin Psychiatry, 78(4):e415-e419 (2017)) Janssen is developing an intranasal delivery product of S-ketamine (Esketamine), currently in Phase 3 (Jansson's media release). Intravenous (IV) administration of ketamine, which is being widely used for off-label treatment of depression, presents numerous challenges, including high clinical delivery cost, inconvenience in drug administration. An intranasal formulation of the S-enantiomer of ketamine has many issues as well, including high variability in use or in absorption among subjects, risks of developing nasal rhinitis due to frequent intranasal administration (Singh et al., Am J Psychiatry, 173(8):816-26 (2016)).

Based on available clinical data, the ketamine films herein can be applied to a subject in need, either alone or in combination with another ketamine formulation (e.g., oral, injection, transdermal patch, etc.) to achieve a desired pharmacological effect with acceptable side effects.

In some specific embodiments, the present disclosure provides a method of treating pain, e.g., break-through cancer pain (BTCP), complex regional pain syndrome (CRPS), refractory cancer pain, neutopathic pain, post traumatic syndrome (PTSD) and/or ischaematic limb pain, the method comprising applying one or more films (e.g., QR, SR, bilayer, or one-directional films) herein to a subject in need, e.g., to an oral cavity of the subject, such as lingual, sublingual, and/or buccal cavity. In some embodiments, the present disclosure also provides a method of treating acute or chronic pain, the method comprising applying one or more films (e.g., QR, SR, bilayer, or one-directional films) herein to a subject in need, e.g., to an oral cavity of the subject, such as lingual, sublingual, and/or buccal cavity. The methods herein are not limited to the forms of pain. For example, the films herein can also be used for acute and chronic pain management due to battlefield wounds. In addition, the film herein by itself or in conjunction with another pain treatment (such as another ketamine formulation/delivery device) can also serve as an alternative to opioids in for treatment of all types of pain. Given the epidemic crisis of opioid use in the U.S. and across the world, any alternative, which can offer similar efficacy with less addiction potential, will have considerable medical, social and commercial value.

In some embodiments, the present disclosure also provides a method of treating one or more diseases or disorders selected from pain (e.g., neuropathic pain, complex regional pain syndrome (CRPS), chronic pain), depression (major depressive disorder, treatment-resistant depression, bipolar depression), restless legs syndrome, a condition associated with spinal cord injury (e.g., autonomic dysreflexia, immune suppression, chronic central neuropathic pain suffering from spinal cord injury, leukocyte apoptosis, splenic atrophy, leucopenia, or combinations thereof), anxiety, bipolar disorder (e.g., childhood-onset bipolar disorder, bipolar depression), stress-induced disorder (e.g., stress-induced affective disorder, stress-induced psychopathology), post-traumatic stress disorder, Alzheimer's dementia, amyotrophic lateral sclerosis, and suicidality, the method comprising applying one or more films (e.g., QR, SR, bilayer, or one-directional films) herein to a subject in need, e.g., to an oral cavity of the subject, such as lingual, sublingual, and/or buccal cavity.

In some embodiments, the films herein can be selected for the methods herein to achieve optimal efficacy and minimal side effects. Those skilled in the art can choose appropriate dosage levels based on available clinical data on ketamine, some of which are exemplified herein, e.g., for the treatment of major depressive disorders (MDD) and pain.

In some embodiments, provided herein is a method of treating pain (e.g., a neuropathic pain) in a subject in need thereof. In such embodiments, the method can comprise administering one or more films herein to achieve a plasma concentration for a desired period of time in the subject ranging from about 50 ng/ml to about 1000 ng/ml, and preferably from about 100 ng/ml to about 1000 ng/ml or from about 300 ng/ml to about 1000 ng/ml, such as about 500 ng/ml.

In some embodiments, provided herein is a method of treating depression (e.g., a major depressive disorder) in a subject in need thereof. In such embodiments, the method can comprise administering one or more films herein to achieve a plasma concentration for a desired period of time in the subject ranging from about 10 ng/ml to about 200 ng/ml, preferably, ranging from about 20 ng/ml to about 100 ng/ml.

A biphasic pharmacokinetic profile can be advantageous in some situations, e.g., to achieve a fast on-set and sustained analgesia or antidepressant effect. For example, the acute pain arising from wounds from battlefield or an accident needs a fast on-set of analgesia, thus rapid ketamine absorption. Since ketamine has a short half-life (˜2-3 hours), maintenance of pain management over hours or days needs sustained exposure of ketamine following the fast absorption phase.

In some embodiments, the methods herein can comprise applying a bilayer film herein with a QR and SR film laminated together that can provide a biphasic PK profile. In some embodiments, the method can comprise initially applying one or more QR films herein, e.g., to the sublingual mucosa of the subject to achieve a fast on-set, e.g., fast analgesia or antidepressant effect, and applying one or more SR films afterwards, e.g., to the buccal mucosa of the subject to achieve a sustained effect, e.g., analgesia or antidepressant effect. For convenience of such methods, a multiple-film package can be provided, which contains one or more QR and SR films as needed.

Ketamine can be extensively metabolized with the major metabolites being norketamine and hydroxyketamines, dehydronorketamine, and hydroxynorketamines. See e.g., Zanos, P. et al., “NMDAR inhibition—independent antidepressant actions of ketamine metabolites,” Nature, 533(7604):481-6(2016), and U.S. Pat. No. 9,650,352. Further, deuterated ketamines (e.g., with at least one of the hydrogen atoms in ketamine being substituted by deuterium above its natural abundance, e.g., greater than 10% deuterium, or more than 90% deuterium) and deuterated norketamines (e.g., with at least one of the hydrogen atoms in norketamine being substituted by deuterium above its natural abundance, e.g., greater than 10% deuterium, or more than 90% deuterium) have been synthesized. See e.g., WO 2017/180589, US Pub. No. 2017/0355663, and U.S. Pat. No. 7,638,651.

In some embodiments, the present invention also provides a ketamine metabolite film (QR or SR film) comprising one or more metabolites chosen from norketamine, hydroxyketamines, dehydronorketamine, and hydroxynorketamines. In some embodiments, the ketamine metabolite film can be essentially the same as any of the respective ketamine film (e.g., QR film, SR film, bilayer film, etc.) described herein, except that the ketamine is partially or completely substituted with one or more metabolites chosen from norketamine, hydroxyketamines, dehydronorketamine, and hydroxynorketamines. For example, in some embodiments, the ketamine metabolite film can be a QR or SR film.

In some embodiments, the active ingredient in the ketamine metabolite film comprises, consists essentially of, or consists of hydroxynorketamine (“HNK”). In some embodiments, the HNK can be in the form of 2R,6R-isomer, 2S,6S-isomer, or any mixtures thereof. In some embodiments, the ketamine metabolite film comprises a therapeutically effective amount of 2R,6R-hydroxynorketamine and is substantially free of 2S,6S-hydroxynorketamine (e.g., the ratio of the 2R,6R-isomer to the 2S,6S-isomer is more than 10:1, more than 20:1, or above). In some embodiments, the ketamine metabolite film can also be used for the treatment of any of the diseases or disorders described herein, for example, depression (e.g., major depressive disorder, treatment-resistant depression, bipolar depression), anxiety, pain (e.g., neuropathic pain, complex regional pain syndrome (CRPS), chronic pain), etc.

In some embodiments, the present invention also provides a deuterated ketamine film comprising one or more of the deuterated analogs chosen from deuterated ketamines and deuterated norketamines. In some embodiments, the deuterated ketamine film is essentially the same as any of the respective ketamine film (e.g., QR, SR, or bilayer film) described herein, except that the ketamine is partially or completely substituted with one or more of the deuterated analogs chosen from deuterated ketamines and deuterated norketamines. For example, in some embodiments, the deuterated ketamine film can be a QR, SR, or a bilayer film. In some embodiments, the deuterated ketamine film can comprise one or more deuterated analogs, one or more permeation enhancers, and other suitable ingredients as described herein. In some embodiments, the active ingredient in the deuterated ketamine film comprises, consists essentially of, or consists of a deuterated ketamine. In some embodiments, the deuterated ketamine film can also be used for the treatment of any of the diseases or disorders described herein, for example, depression (e.g., major depressive disorder, treatment-resistant depression, bipolar depression), anxiety, pain (e.g., neuropathic pain, complex regional pain syndrome (CRPS), chronic pain), etc.

Definitions

As used herein, the term “about” modifying an amount related to the invention refers to variation in the numerical quantity that can occur, for example, through routine testing and handling; through inadvertent error in such testing and handling; through differences in the manufacture, source, or purity of ingredients employed in the invention; and the like. As used herein, “about” a specific value also includes the specific value, for example, about 10% includes 10%. Whether or not modified by the term “about”, the claims include equivalents of the recited quantities. In one embodiment, the term “about” means within 20% of the reported numerical value.

It will be understood that the term “film” includes thin films and sheets, in any shape, including rectangular, square, or other desired shape. The films described herein can be any desired thickness and size such that it can be placed into the oral cavity of the user. For example, the films can have a relatively thin thickness of from about 0.1 to about 10 mils, or they can have a somewhat thicker thickness of from about 10 to about 30 mils. For some films, the thickness can be even larger, i.e., greater than about 30 mils. Films can be in a single layer or they can be multi-layered, including laminated films.

In various embodiments, the oral dissolving films herein can be categorized as fast dissolving (alternatively quick-releasing or simply quick release or QR), or slow dissolving (alternatively slow-releasing or simply slow release or SR). Fast dissolving films generally dissolve in about 1 second to about 30 minutes in the mouth, and slow dissolving films generally dissolve in more than 30 minutes in the mouth. In some embodiments, the QR films or the SR films can be characterized by certain in vitro dissolution profile, such as those described herein.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

The term “therapeutically effective amount,” as used herein, refers to that amount of a therapeutic agent (e.g., ketamine) sufficient to result in amelioration of one or more symptoms of a disorder or condition (e.g., pain, depression), or prevent appearance or advancement of a disorder or condition, or cause regression of or cure from the disorder or condition.

The term “subject” (alternatively referred to herein as “patient”) as used herein, refers to an animal, preferably a mammal, most preferably a human, who has been the object of treatment, observation or experiment.

### EXAMPLES

### Example 1

### Fast-Dissolving Oral Film

This example shows a fast-dissolving oral film prepared with a highly water-soluble polymer such as PVP (for example Kolidon 90F) as the film matrix polymer. Water solubility and film dissolution of the film could be adjusted with a modified polymer such as a copolymer of PVP-vinyl acetate (for example, Kolidone VA 64). The formulation is given in Table 1.

TABLE 1
Formulation composition of Example 1

| Ingredient | Function | Wet grams | Dry Parts | Dry % |
|---|---|---|---|---|
| Water | Solvent | 4 | | |
| Ethanol | solvent | 30 | | |
| Kollidon 90F | Film | 8 | 8 | 53.6% |
| | polymer | | | |
| Kollidon VA64 | Film | 2 | 2 | 13.4% |
| | polymer | | | |
| Ketamine Free | Active | 2.25 | 2.25 | 15.07% |
| Base | ingredient | | | |
| Tocopherol | antioxidant | 0.08 | 0.08 | 0.54% |
| acetate | | | | |
| PEG 400 | plasticizer | 0.8 | 0.8 | 5.4% |
| Oleic alcohol | Permeation | 1.5 | 1.5 | 10.0% |
| | enhancer | | | |
| Propylene Glycol | plasticizer | 0.3 | 0.3 | 2.0% |
| Total | | 48.93 | 14.93 | 100% |

The unit dose was tested for dissolution rate with USP apparatus 2 and was found to dissolve quickly; 85% of the dose could be released in about 5 minutes.

### Example 2

### Sustained-Release Oral Film

This example demonstrates an oral film of ketamine with higher muco-adhesion and slower drug release property that can be prepared with a combination of two water-swellable polymers such as Carbopol (polyacrylic acid, supplied by Lubrizol) and sodium carboxylmethyl cellulose (Na CMC). Carbopol provides strong adhesion by ionic interaction when wetted with saliva, on the oral mucosal tissues. The formulation is given below (Table 2).

TABLE 2
Formulation composition of Example 2

| Ingredient | wet g | dry g | Dry% |
|---|---|---|---|
| Propylene Glycol | 4.04 | 4.0 | 18.34862 |
| PEG 400 | 3.04 | 3.0 | 13.76147 |
| Ketamine free base | 3.63 | 3.6 | 16.51376 |
| Sucralose | 0.14 | 0.14 | 0.642202 |
| Oleic acid | 1.10 | 1.10 | 5.045872 |
| Acetone | 16.03 | — | |
| Ethanol | 16.00 | — | |
| Purified Water | 27.99 | — | |
| HPMC E5 | 5.61 | 5.6 | 25.68807 |
| Na CMC Type 12 | 3.47 | 3.5 | 16.05505 |
| Carbopol 971P | 0.74 | 0.7 | 3.211009 |
| Total | 81.79 | 21.8 | |

The unit dose was tested for in vitro permeation rate and was found to penetrate through the cultured human buccal tissue in about 6 hours, indicating a sustained drug release property (FIG. 3). Cultured buccal tissues are purchased from MatTek Corporation. MatTek's EpiOral tissues consist of normal, human-derived oral epithelial cells. The cells have been cultured to form multilayered, highly differentiated models of the human buccal (EpiOral) and gingival (EpiGingival) phenotypes. The tissues are cultured on specially prepared cell culture inserts using serum free medium and attain levels of differentiation on the cutting edge of in vitro cell culture technology. The EpiOral tissue models exhibit in vivo-like morphological and growth characteristics which are uniform and highly reproducible. Permeation experiments were conducted using MatTek's Protocol MK-24-007-0014, “Drug Absorption Protocol for EpiOral Tissue Model (ORL-212-PERC & ORL-606-PERC)”, November 2014.

### Example 3

### One-Dimensional Released Bi-Layered Film

The Formulation prepared here has two layers: the mucosal adhesive layer (Example B) and an ingestible layer formulated with a polymethacrylate (Eudragit)-based polymer (Evonik), which is commonly used as a tablet film coating for solid oral formulations. The bilayer configuration was prepared by two-casting processes with the ingestible layer formulation shown in Table 3.

TABLE 3
Formulation composition of Example 3

| Ingredient | actual, g | Dry g | Dry % |
|---|---|---|---|
| Ethanol | 43.00 | | |
| Water | 10.00 | | |
| Eudragit L100-55 | 11.44 | 11.440 | 45.40% |
| Eudragit RL PO | 7.36 | 7.360 | 29.21% |
| Titanium dioxide | 1.00 | 1.000 | 3.97% |
| PEG400 | 4.21 | 4.000 | 15.87% |
| Propylene glycol | 1.80 | 1.400 | 5.56% |
| Total | 78.81 | 25.20 | 100% |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A slow-release film composition comprising ketamine in a mucoadhesive polymer, preferably, when tested in vitro using cultured human buccal tissue in a Franz model, the film composition delivers ketamine through the human buccal tissue at a rate of about 0.1 mg/cm2*h to about 5 mg/cm2*h (e.g., about 0.1 mg/cm2*h to about 2 mg/cm2*h) for the first hour, first two hours, or first four hours.

2. The slow-release film composition of claim 1, when tested in vitro using cultured human buccal tissue in a Franz model, delivers ketamine through the human buccal tissue at a rate of about 0.1 mg/cm2*h to about 1 mg/cm2*h for the first hour, first two hours, or first four hours.

3. The slow-release film composition of claim 1, when tested in vitro using cultured human buccal tissue in a Franz model, delivers ketamine through the human buccal tissue at a rate of about 0.2 mg/cm2*h to about 0.5 mg/cm2*h for the first hour, first two hours, or first four hours.

4. The slow-release film composition of claim 1, wherein the ketamine is present in an amount of about 5% to about 30% by weight of the film composition.

5. The slow-release film composition of claim 1, wherein the mucoadhesive polymer comprises one or more polymers selected from cellulose derivatives, polyacrylic acids, polyacrylates, polyethylene oxides, polyvinyl pyrrolidones, polyvinyl alcohols, tragacanth, alginates, gum (including karaya gum, guar gum, xanthan gum), soluble starch, gelatin, lectin, pectin, and chitosan.

6. The slow-release film composition of claim 1, wherein the mucoadhesive polymer comprises one or more polymers selected from a hydrophilic polymer, a polysaccharide and its derivatives, and a hydrogel.

7. The slow-release film composition of claim 1, wherein the mucoadhesive polymer comprises one or more polymers selected from polyacrylic acids, polyacrylates, celluloses, e.g., carboxy celluloses (e.g., sodium carboxymethyl cellulose), hydroxyalkyl cellulose (e.g., hydroxypropylcellulose, hydroxy ethylcellulose and hydroxy ethyl ethyl cellulose)), polyvinylpyrrolidone, and polyvinyl alcohol.

8. The slow-release film composition of claim 1, wherein the mucoadhesive polymer comprises one or more polymers selected from Carbopol (polyacrylic acid), carboxymethyl cellulose, carboxyethyl cellulose, hydroxypropyl cellulose, hydroxypropylmethyl cellulose, and gum.

9. The slow-release film composition of claim 1, wherein the mucoadhesive polymer is water-swellable.

10. The slow-release film composition of claim 1, wherein the mucoadhesive polymer is present in an amount of about 15% to about 60% by weight of the film composition.

11. The slow-release film composition of claim 1, further comprising a permeation enhancer, e.g., comprising one or more permeation enhancers selected from dimethyl sulfoxide (DMSO), oleic alcohol, oleic acid, oleyl oleate, levulinic acid, propylene glycol, dipropylene glycol, ethanol, and surfactants.

12. The slow-release film composition of claim 11, wherein the permeation enhancer is present in an amount of about 2% to about 20% by weight of the film composition.

13. The slow-release film composition of claim 1, further comprising an antioxidant.

14. The slow-release film composition of claim 13, wherein the antioxidant is tocopherol acetate.

15. The slow-release film composition of claim 1, wherein the ketamine is substantially in its free base form.

16. A one-directional release film composition comprising a mucoadhesive layer and a water-insoluble but ingestible layer,
wherein the mucoadhesive layer is or comprises a slow-release film composition of claim 1.

17. The one-directional film composition of claim 16, wherein the water insoluble but ingestible layer comprises a cellulose (e.g., ethyl cellulose), a poly acrylate (e.g., polymethylacrylate), or a combination thereof.

18. The film composition of claim 1, suitable for use to deliver ketamine through an oral mucosal membrane, e.g., sublingual mucosal or buccal mucosal membrane.

19. A composition comprising:
a) a quick-release film comprising ketamine and a water soluble polymer; and
b) a slow-release film comprising ketamine and a water-swellable polymer.

20. The composition of claim 19, wherein the quick-release film is characterized in that it releases substantially all of the ketamine therein in less than 30 minutes, when tested in vitro in a dissolution test using USP Apparatus 2.

21. The composition of claim 19, wherein the water soluble polymer in the quick-release film comprises one or more polymers selected from hydroxyl propyl methyl cellulose (HPMC), hydroxylpropyl cellulose (HPC), Povidone, polyvinyl alcohols (PVA), low molecular weight polyethylene oxide (PEO such as PolyOx N10 supplied by Dow Chemical), and starch-based polymers (Lycoat, manufactured by Roquette).

22. The composition of claim 19, wherein the water-swellable polymer in the slow-release film comprises one or more polymers selected from Carbopol, high molecular weight PEO, xanthan gum, and chitosan.

23. The composition of claim 19, wherein, when tested in an in vitro release test, the composition releases ketamine at a rate of at least 0.5 mg/cm2*h (e.g. , about 1 mg/cm2*h to about 20 mg/cm2*h) for the first two hours, and releases ketamine at a rate of about 0.5 mg/cm2*h to about 5 mg/cm2*h thereafter for an extended period of about 2 hours, 4 hours, 6 hours, or 8 hours.

24. The composition of claim 19, wherein the quick-release film and slow-release film are laminated to form a single unit dosage form.

25. The composition of claim 19, wherein the quick-release film and/or slow-release film further comprises a permeation enhancer.

26. The composition of claim 25, wherein the permeation enhancer comprises one or more permeation enhancers selected from dimethyl sulfoxide (DMSO), oleic alcohol, oleic acid, oleyl oleate, levulinic acid, propylene glycol, dipropylene glycol, ethanol, and surfactants.

27. The composition of claim 25, wherein the permeation enhancer is present in an amount of about 2% to about 20% by weight of the quick-release film or slow-release film, as applicable.

28. The composition of claim 19, wherein the quick-release film and/or slow-release film further comprises an antioxidant.

29. The composition of claim 28, wherein the antioxidant is tocopherol acetate.

30. The composition of claim 19, wherein the quick-release film and/or slow-release film comprises ketamine in substantially its free base form.

31. A method of administering ketamine to a subject in need thereof, the method comprising administering to an oral mucosal membrane of the subject the film composition of claim 1.

32. A method of administering ketamine to a subject in need thereof, the method comprising a) administering to the subject sublingually a quick release film comprising ketamine and a water soluble polymer; and b) subsequently, administering to the subject buccally a slow-release film comprising ketamine and a water-swellable polymer, wherein the administration generates a biphasic pharmacokinetic profile, which comprises a fast-rising ketamine plasma concentration profile followed by a slow-rising and sustained ketamine plasma concentration profile.

33. The method of claim 31, wherein the subject suffers from depression and/or pain.

34. A method of treating depression and/or pain in a subject in need thereof, the method comprising administering to an oral mucosal membrane, e.g., sublingual mucosal or buccal mucosal membrane, of the subject the film composition of claim 1.

35. A kit comprising
a) a quick-release film comprising ketamine and a water soluble polymer; and
b) a slow-release film comprising ketamine and a water-swellable polymer.
